(19) United States
(12) Patent Application Publication
(10) Pub. No.: US 2014/0243853 A1
(43) Pub. Date: Aug. 28, 2014

(54) SUTURE CUTTING AND REMOVAL TOOL

(71) Applicant: John Temple, Chelsea, MI (US)

(72) Inventor: John Temple, Chelsea, MI (US)

(21) Appl. No.: 13/775,372

(22) Filed: Feb. 25, 2013

(51) Int. Cl. A61B 17/04

(52) U.S. Cl. CPC A61B 17/0467
USPC 606/138

(57) ABSTRACT

An improved, versatile suture cutting and removal tool comprises an elongated, hand-held body terminating in a distal end section including an upwardly oriented suture-cutting blade. Right and left tweezers on opposing side of the blade grasp and remove suture material once cut by the blade. In the preferred embodiment, the right and left tweezers are independently controlled by respective right and left depressible buttons. In particular, the right and left tweezers may include inclined ramp surfaces, with the right and left tweezers being independently controlled by respective right and left depressible buttons having respective inclined ramp surfaces that engage with the ramp surfaces of the tweezers. The blade is preferably supported on a central member having opposing right and left side edges, with the right and left tweezers each including an edge that bears against one of the edges of the central member to grasp and remove suture material.

### FIELD OF THE INVENTION

This invention relates generally to suture removal and, in particular, to a versatile suture cutting and removal tool.

### BACKGROUND OF THE INVENTION

There are numerous implements design to cut and/or remove sutures. However, most of these tools are either awkward to use, do not facilitate removal from both sides of a cutting line, or present both of these or other deficiencies.

### SUMMARY OF THE INVENTION

This invention resides in an improved, versatile suture cutting and removal tool. The tool comprises an elongated, hand-held body terminating in a distal end section. The distal end section includes an upwardly oriented suture-cutting blade, and right and left tweezers on opposing side of the blade are provided to grasp and remove suture material once cut by the blade.

In the preferred embodiment, the right and left tweezers are independently controlled by respective right and left depressible buttons. In particular, the right and left tweezers may include inclined ramp surfaces, with the right and left tweezers being independently controlled by respective right and left depressible buttons having respective inclined ramp surfaces that engage with the ramp surfaces of the tweezers.

The blade is preferably supported on a central member having opposing right and left side edges, with the right and left tweezers each including an edge that bears against one of the edges of the central member to grasp and remove suture material.

The tool is constructed of plastic, wherein the right and left tweezers open and close through separate plastic flexion areas or living hinges. The right and left tweezers may be independently controlled by respective right and left depressible buttons, with the buttons also operating through plastic flexion areas or living hinges.

The distal end portion may include a bottom surface adapted to slide against a region of skin having a suture to be removed with the body being inclined upwardly, and wherein the right and left tweezers open and close in a plane parallel to the bottom surface.

### BRIEF DESCRIPTION OF THE DRAWINGS

FIG. 1 is a top-down view of a preferred embodiment of the invention;

FIG. 2 is a bottom view;

FIG. 3 is a side view;

FIGS. 4A and 4B are oblique views illustrating the way in which the tool is gripped and used; and

FIGS. 5A-5C are front views of the distal end portion perhaps better depicting the scissoring action.

### DETAILED DESCRIPTION OF THE INVENTION

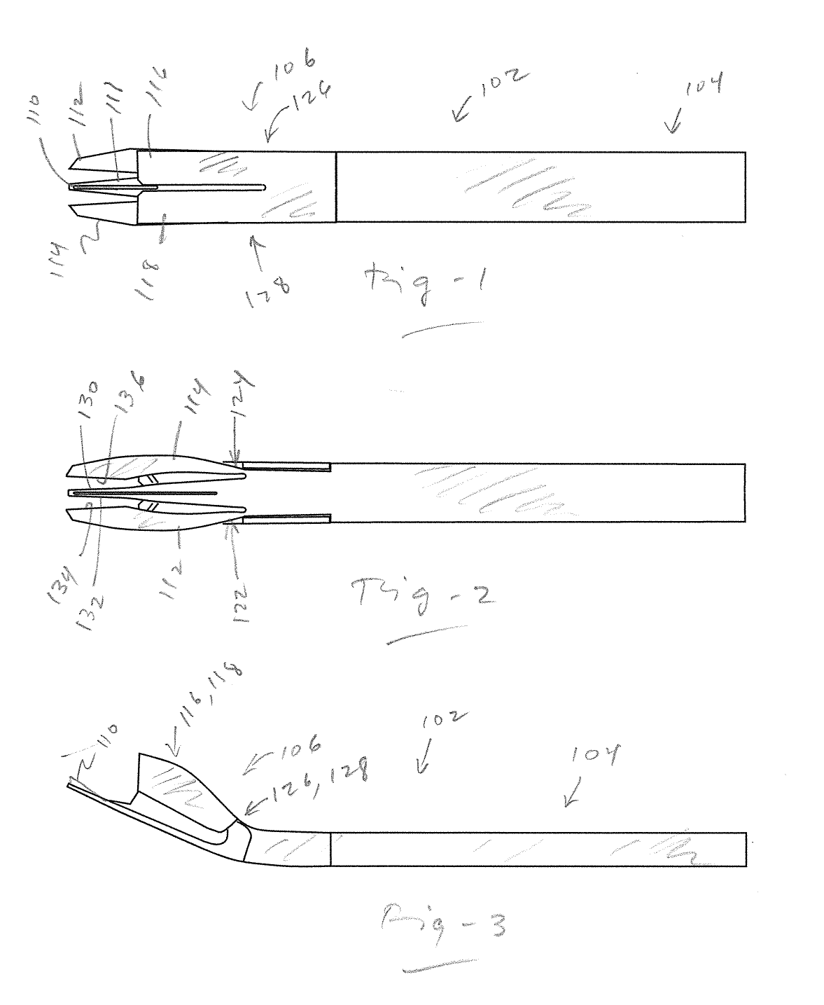

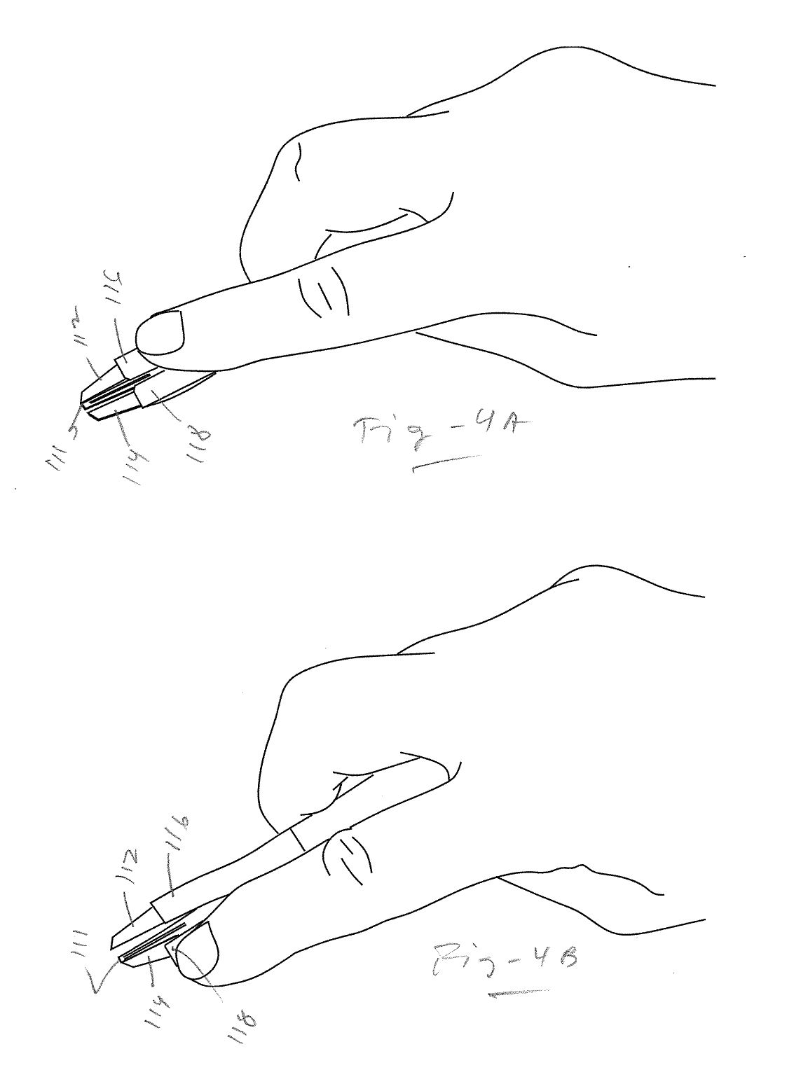

FIG. 1 is a top-down view of a preferred embodiment of the invention. FIG. 2 is a bottom view and FIG. 3 is a side view. The tool comprises an elongate body 102 having a proximal gripping section 104 and a distal end section 106. The distal end section includes an upwardly oriented suture-cutting blade 110 and right and left tweezer members 112, 114 on opposing side of the blade to grasp and remove suture material once cut by the blade 110. The blade 110 is mounted to a central, elongated member 111.

In the preferred embodiment, the right and left tweezer members are independently controlled by respective right and left depressible buttons 116, 118. With the exception of blade 110, the tool is composed of plastic, and the tweezer members and button move due to the inherently flexible nature of the material and or through the action of “ living hinges.” In particular, tweezer member 112 flexes at 122, tweezer member 114 flexes at 124, button 116 flexes at 126, and button 118 flexes at 128.

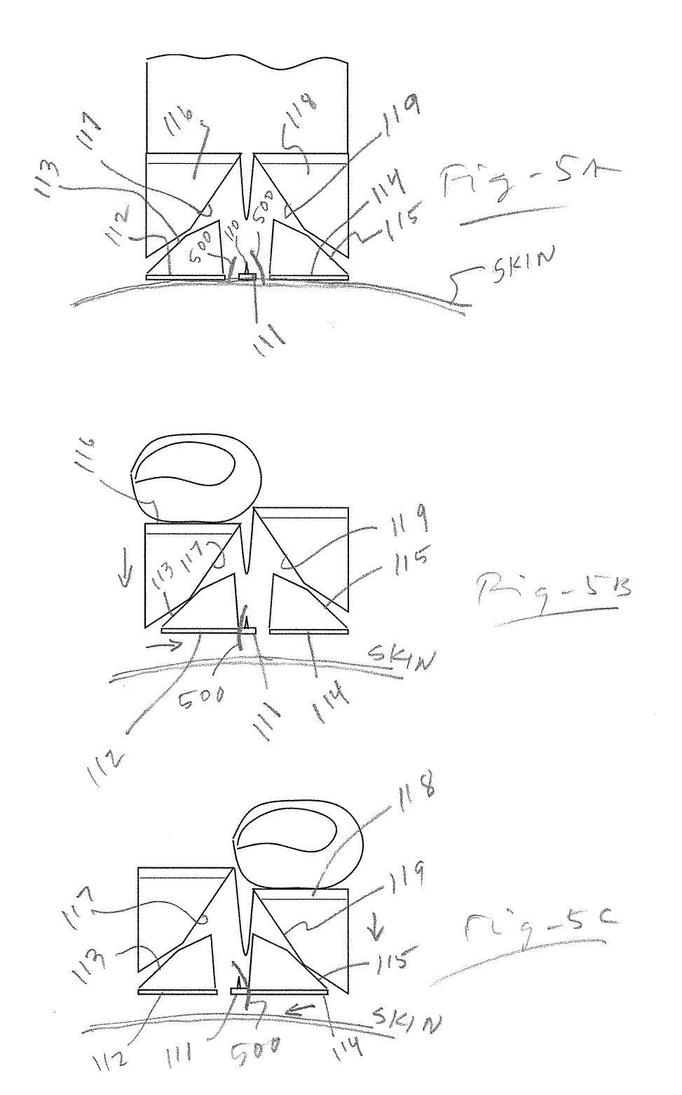

With reference to FIG. 2, the central, blade-holding member 111 has side edges 130, 132. Scissor member 112 has an edge 134, and scissor member 114 has an edge 136. During use, the edges of the scissor members cooperate with the edges of the blade-holding member 111 to pull out suture material as described with reference to FIG. 5. Some or all of the suture-grasping edges of the scissor members and the blade-holding member may be serrated or otherwise roughened for enhanced gripping power.

FIGS. 4A and 4B are oblique views illustrating the way in which the tool is gripped and used. In FIG. 4A, a user has depressed button 116, causing the inner edge of scissor member 112 to close against the right edge of member 111. In FIG. 4B, the user has depressed button 118, causing the inner edge of scissor member 114 to close against the left edge of member 111. Note that in accordance with the invention the user uses the same hand to hold the tool to remove suture material on either side of the blade, using the same finger to press either button 116, 118.

FIGS. 5A-5C are front views of the distal end portion perhaps better depicting the scissoring action. In FIG. 5A, a suture 500 has been cut by blade 110 mounted to member 111. The bottom portions of the scissor members 112, 114 and central blade-holding member 111 typically rest against the skin during the suture-cutting operation. Note that the right button 116 includes downwardly and inwardly facing ramps 117, and 119, and that the scissor members include upwardly and inwardly facing ramps 113, 115. It is the cooperation between these ramp surfaces that causes the scissor members to close when the buttons are depressed. Although the various ramps are shown having piecewise linear surfaces, they may be flat, or curved, or at different angles than those measurable from the drawiings so long as the operation described are fulfilled.

In FIG. 5B, with the suture material 500 now severed, button 116 has been depressed, causing ramp 117 to slide against ramp 113, thereby moving tweezer member 112 against central member 111, allowing the user to pull out the suture 500 and away from the skin. In FIG. 5C, with the suture material 500 now severed, button 118 has been depressed, causing ramp 119 to slide against ramp 115, thereby moving tweezer member 114 against central member 111, allowing the allows the user to pull out the suture 500 and away from the skin on the opposite side. The choice of which side to user is up to the user based upon such considerations as the length of the available material on one side or the other, the location of suture knots, and so forth. Note that as either button is depressed the central member 111 may move slight left to right which is not a problem due to the resilient nature of the plastic material used for construction.

1. A suture cutting and removal tool, comprising:
an elongated, hand-held body terminating in a distal end section;
the distal end section including an upwardly oriented suture-cutting blade; and
right and left tweezers on opposing side of the blade to grasp and remove suture material once cut by the blade.

2. The suture cutting and removal tool of claim 1, wherein the right and left tweezers are independently controlled by respective right and left depressible buttons.

3. The suture cutting and removal tool of claim 1, wherein:
the right and left tweezers include inclined ramp surfaces; and
the right and left tweezers are independently controlled by respective right and left depressible buttons having respective inclined ramp surfaces that engage with the ramp surfaces of the tweezers.

4. The suture cutting and removal tool of claim 1, wherein:
the blade is supported on a central member having opposing right and left side edges; and
the right and left tweezers each include an edge that bears against one of the edges of the central member to grasp and remove suture material.

5. The suture cutting and removal tool of claim 1, wherein:
the tool is constructed of plastic; and
the right and left tweezers open and close through separate plastic flexion areas or living hinges.

6. The suture cutting and removal tool of claim 1, wherein:
the right and left tweezers are independently controlled by respective right and left depressible buttons; and
the buttons operate through plastic flexion areas or living hinges.

7. The suture cutting and removal tool of claim 1, wherein:
the distal end portion includes a bottom surface bottom surface adapted to slide against a region of skin having a suture to be removed with the body being inclined upwardly; and
the right and left tweezers open and close in a plane parallel to the bottom surface.

8. A suture cutting and removal tool, comprising:
an elongated, hand-held body terminating in a distal end section with a bent portion between the body and the end section;
the end section having a substantially flat bottom surface adapted to move relative to a region of skin having a suture to be removed with the body being inclined upwardly;
the distal end section further including a central, forward-oriented prong with right and left opposing sides and an upwardly oriented suture-cutting blade;
a right movable tweezer portion having a right inward edge and a right upper ramp surface oriented upwardly and inwardly toward the end section;
a left movable tweezer portion having a left inward edge and a left ramp surface oriented upwardly and inwardly toward the end section;
a right depressible button having a right lower ramp surface oriented downwardly and inwardly and with an opposing facing relation to the ramp surface of the right tweezer portion;
a left depressible button having a left lower ramp surface oriented downwardly and inwardly and with an opposing facing relation to the ramp surface of the left tweezer portion; whereby:
when the right button is pressed, the right lower ramp surface engages with the right upper ramp surface, causing the right inward edge to close against the right side of the central prong, and
when the left button is pressed, the left lower ramp surface engages with the left upper ramp surface, causing the left inward edge to close against the left side of the central prong.
